(19) United States
(12) Patent Application Publication
(10) Pub. No.: US 2024/0285540 A1
(43) Pub. Date: Aug. 29, 2024

(54) FUNCTIONAL THERAPEUTIC INDEX IN THE TREATMENT OF PERIPHERAL IMMUNE DYSFUNCTION

(71) Applicant: Desmond Mascarenhas, Auburn, CA (US)

(72) Inventor: Desmond Mascarenhas, Auburn, CA (US)

(21) Appl. No.: 18/407,753

(22) Filed: Jan. 9, 2024

Related U.S. Application Data
(63) Continuation of application No. PCT/US23/13908, filed on Feb. 26, 2023.

(51) Int. Cl. A61K 9/50; A61K 9/10; A61K 38/40; A61K 45/06; A61K 47/36; C07K 14/00

(52) U.S. Cl. CPC A61K 9/5031; A61K 9/10; A61K 38/40; A61K 45/06; A61K 47/36; C07K 14/001

(57) ABSTRACT

Therapeutic index (TI) is a measure of drug safety based on the differential between minimal effective dose and maximal tolerable dose. Many small-molecule drugs show limited TI (below 10). Given patient variability, such a limited range can become further narrowed for a drug in practice, complicating its real-world use. Epigenetic drugs such as nuclear receptor ligands (NRLs)—which must reach the target cell's nucleus in order to function—pose a particular challenge because small-molecule drugs typically lack the evolved biological “intelligence” that natural molecules employ in order to reach their intracellular targets efficiently. Methods and compositions are disclosed herein for the treatment of peripheral immune tissues (PIT) using NRLs covalently attached to immodulin peptides. PIT includes surface draining lymph nodes of the upper and lower limbs and adjacent skin tissues. Dramatic improvements in the TI of NRLs can be gained from the immodulin scaffold's anatomic specificity (natural homing to PIT), precise organelle targeting (efficient cellular uptake and nuclear transport), and tunable mechanism of transcriptional action (immodulins themselves drive intranuclear RXR heterodimer formation naturally, thereby placing NRLs in close proximity to their RXR-heterodimer targets). The combined effect of such improvements is a 2-100-fold improvement in functional TI for small-molecule NRL drugs. This invention discloses three key components of that observed improvement: a preferential anatomical distribution of immodulins to the peripheral immune system when administered via a transdermal route, addition of hyaluronic acid to the diluent to enhance homing, and the novel use of transferrin for enhancing NRL-liganded immodulin recovery from a polymer-protected transdermal formulation.

### RELATED APPLICATIONS

This invention claims priority to PCT/US2023/013908 with an international filing date of 26 Feb. 2023, which claims benefit of international application number PCT/US2022/018449 with an international filing date of 2 Mar. 2022, which claims benefit of international application number PCT/US2021/046814 with an international filing date of 20 Aug. 2021 and international application number PCT/US2021/021433 with an international filing date of 9 Mar. 2021. PCT/US2021/021433 claims priority to international application number PCT/US2020/024828 with an international filing date of 26 Mar. 2020.

### TECHNICAL FIELD

This invention relates to the field of guided therapeutics, and more particularly to the use of ligand-modified immodulin peptides to deliver small-molecule transcriptional modulators to their destinations with increased precision. The compositions and methods herein provide modified immodulin peptides that serve as versatile scaffolds that offer greater mechanistic, anatomical and temporal precision of action, leading to safer and more specific therapeutics. In light of their adjustable mechanistic specificity, immodulins are powerful new tools in re-purposing small-molecule transcriptional modulators, endowing them with greater anatomical precision, bioavailability and safety, particularly when administered by the routes and formulations herein disclosed. Such agents may have particular relevance in the burgeoning fields of distance medicine and self-administered therapeutics.

### BACKGROUND AND SUMMARY OF THE INVENTION

Therapeutic index (TI) is a measure of drug safety based on the differential between minimal effective dose and maximal tolerable dose. Many small-molecule drugs show limited TI (below 10). Given patient variability, such a limited range can become further narrowed for a drug in practice, complicating its real-world use. Epigenetic drugs such as nuclear receptor ligands (NRLs)—which must reach the target cell's nucleus in order to function—pose a particular challenge because small-molecule drugs lack the evolved biological “intelligence” that natural molecules employ in order to reach their intracellular targets efficiently. The observed TI of one FDA-approved NLR, bexarotene, is only about 3-4 when used to treat cancer in the lymphatic system. Methods and compositions are disclosed herein for the treatment of peripheral immune tissues (PIT) using NRLs covalently attached to immodulin peptides. Dramatic improvements in the TI of NRLs can be gained from the immodulin partner's tissue specificity (natural homing to PIT), precise organelle targeting (efficient cellular uptake and nuclear transport), and tunable mechanism of transcriptional action (immodulins themselves naturally drive intranuclear RXR heterodimer formation, thereby placing NRLs in close proximity to their RXR-partner targets). The combined effect of such improvements is a 2-100-fold improvement in TI for small-molecule NRL drugs. The combined effect of such improvements is a 2-100-fold improvement in functional TI for small-molecule NRL drugs. This invention discloses key components of that observed improvement: a preferential anatomical distribution of immodulins to the peripheral immune system when administered via a transdermal route, addition of hyaluronic acid to the diluent to enhance homing, and the novel use of transferrin for enhancing NRL-liganded immodulin recovery from a polymer-protected transdermal formulation.

Natural homing of immodulins to skin and surface lymphatics, while maintaining low to undetectable levels in general circulation, appears to be an active sequestration phenomenon, rather just a proximity phenomenon from transdermal administration. In support of this claim, Examples show that when the site of transdermal administration is the forearm (upper limb), high concentrations of fluorescent-labeled immodulin accumulate in calf skin and draining lymph node (popoliteal) of the lower limb as well, with much lower concentrations in plasma. Conversely, when applied transdermally to calf skin, high concentrations of immodulin are also found in skin and draining lymph nodes (brachial) of the upper limb—again without generating comparable levels in plasma, blood cells or mesenteric lymph nodes. This type of biodistribution has not, to the inventor's knowledge, ever been reported for an epigenetic drug.

Immodulin peptides and ligand modifications thereof are described in PCT/US2023/013908, PCT/US2022/018449, PCT/US2021/046814, PCT/US2021/021433 and PCT/US2020/024828, as well as in a recent scientific publication (see Mascarenhas, 2022; and references cited therein). The term ‘precision’, as used herein, refers to improved mechanistic, anatomical and temporal precision in the administration of medicine.

This invention provides compositions and methods that substantially increase anatomic specificity of therapeutics to the peripheral immune tissues. As disclosed here for the first time, native immodulin peptides possess a capability for targeting these immune tissues, especially surface lymphatics of the skin. However, convenient transdermal administration of peptides, especially when trapped in protective polymers such as poly-lactic-co-glycolic acid (PLGA), introduces practical problems in the efficient release of immodulin peptides from the polymer. Here disclosed is the surprising observation that mixing recombinant human holo-transferrin with the immodulin during encapsulation in a PLGA polymer increases release efficiency by more than three-fold. Transferrin is a natural ligand of immodulin peptides (Mascarenhas, 2022). The method of this invention therefore includes a mandatory step wherein transferrin is added to a ligand-modified peptide prior to encapsulation in a polymer. Another natural ligand, hyaluronic acid, is found to enhance efficacy of immodulin-based constructs in a rat burn model. This may reflect improved targeting to lymphatics in vivo. The method of this invention therefore includes the optional inclusion of hyaluronate in the diluent. Also optionally included in the diluent is a novel set of botanical ingredients that combine to enhance the solubility of constituents, preservative characteristics and skin penetration. Thus the invention builds upon the natural qualities of immodulins, amplifying and extending them through novel formulations so as to make them industrially useful. These modifications, in aggregate, can improve functional therapeutic index over traditional therapeutics administered in conventional ways by as much as a factor of 1000% or more. The unusual combination of elements disclosed herein for increasing mechanistic, anatomic and temporal specificity of NRLs is new.

Covalent addition of small molecules to peptides and proteins is widely employed in laboratory research, but can be prohibitively expensive to scale in manufacture, as well as biologically problematic, for example, with respect to immunogenicity. Using a fragment of an abundant natural protein in the human blood as a scaffold for drug development has obvious advantages, such as the presumed benefit of over a billion years of natural evolutionary design providing biological intelligence that small-molecule drugs almost always lack. This type of biological enhancement can dramatically improve lymphatic targeting, protection from degradation in transit, rapid and selective uptake into the appropriate target cells within target organs, and precise transport within cells to the appropriate location for therapeutic action, such as the nucleus of the cell. Nuclear receptor ligands are examples of previously tested small-molecule drugs that can be repurposed with great specificity using the novel technologies disclosed in this invention. The resulting “ligand-modified” immodulin peptides are tunable transcriptional regulators that exquisitely modulate a cell's transcriptional machinery in its response the extracellular cues. They can be administered to a subject using a traditional route and can find their way to the desired target with far greater efficiency than the ligands themselves could. Since immodulin peptides are themselves RXR transcriptional modulators, the actions of ligand and carrier immodulin can be synergistic. Depending on the type of ligand modification employed, therapeutics built on an immodulin peptide scaffold can, for example, kill cancer cells without harming normal cells; or stimulate the differentiation of cell types such as macrophages, T-cells, osteoblasts and myoblasts to differentiate into desired lineages, thereby dramatically affecting natural biological phenomena and disease.

Covalent addition of small-molecule drugs to antibodies (antibody-drug conjugates, or ADCs) has been explored as a method of expanding functional therapeutic index. Therapeutic index (TI) is a ratio that expresses the relationship between the dose expected to elicit some adverse effect (e.g., LD₅₀, TD₅₀, etc.) and the dose needed to elicit therapeutic effects (e.g., ED₅₀). A key part of drug discovery and development is the characterization and optimization of the safety and efficacy of drug candidates to identify those that have an appropriately balanced safety-efficacy profile for a given indication. The therapeutic index (TI)—which is typically considered as the ratio of the highest exposure to the drug that results in no toxicity to the exposure that produces the desired efficacy—is an important parameter in efforts to achieve this balance. Various types of safety and efficacy data are generated in vitro and in vivo (in animals), and these data can be used to predict the clinical TI of a drug candidate at an early stage of development. The expansion of TI is critical to the expanded use of drugs in distance medicine—involving needle-free self-administration of the drug by patients. This fits a powerful current trend in the evolution of healthcare delivery in advanced economies. Needle-free administration using transdermal and intranasal routes is thus particularly relevant to those drugs that cannot be put into a pill.

TI can be expanded by improving efficacy and/or reducing toxicity of the drug. While targeted delivery using conjugates such as antibody-drug conjugates (ADCs) for treating tumors have gained interest based on recent clinical successes showing improved objective response rates and durability of responses, thereby broadening the TI of their payloads, the clinical data suggest that maximum tolerated doses of payloads and conjugates (when dose-normalized for payload) generally remain comparable. Thus, in drug repurposing, the covalent linkage of a targeting agent (e.g. an antibody) may extend TI by enhancing targeting efficacy without improving safety of the drug itself.

A second feature of ADCs is delivery location. Only surface markers (<5% of all druggable targets) can be targeted. If the cellular location for drug action lies inside a particular organelle (such as epigenetic drugs that work in the nucleus) there are many unsolved inefficiencies in platforms such as ADCs that stop working at the cell surface.

Immodulin peptides offer a promising new approach in the repurposing of ligands whose action involves binding to nuclear receptors, a class of transcriptional factors that can generate unique transcriptional programs in nearly every cell type. Hundreds of nuclear receptor ligands are known, many have been tested clinically, and some—like bexarotene and tamibarotene—have been approved for clinical use. Immodulins are binding partners of RXR isoforms alpha and gamma (Mascarenhas, 2022). They enter target cells by active mechanisms that are influenced by extracellular cues. They are quickly translocated into the nucleus. There, they can alter or enhance RXR-driven transcriptional programs. In principle, therefore, they offer better prospects as carriers of small-molecule nuclear receptor ligands (SMNRL) because, unlike antibodies, immodulins enter the desired cell, reach the desired organelle and directly facilitate alterations to the cell's transcriptional programs. Immodulins are ligands of RXR, a key component of the transcriptional complexes affected by SMNRLs (Mascarenhas, 2022). Because hundreds of SMNRLs are commercially available, the possibility of altering any of dozens of RXR heterodimers possessing unique transcriptional functions is dramatically expanded by the immodulin-SMNRL platform. As immodulins are themselves fragments of abundant plasma proteins (insulin-like growth factor-binding proteins, IGFBPs), no platform toxicities are predicted, or observed experimentally so far in rodents.

This invention discloses additional advantages to be gained from using the immodulin-SMNRL platform: biodistribution experiments in rats show striking changes in tissue accumulation of immodulin-SMNRLs using different routes of administration. In particular, samples from the peripheral immune system tissues, such as skin and draining lymph nodes of the skin, show enhanced concentrations of peptide 2 hours after transdermal administration, compared to traditional routes such as subcutaneous bolus injection and intranasal administration. Using hyaluronic acid, an immodulin ligand, as a carrier can accentuate this targeting effect. By calculating the relative concentration of peptide in surface lymphatic tissues, plasma and major abdominal organs such as liver, kidney, heart, gastrointestinal tract, pancreas and lung, ten-fold or more improvement of TI can be predicted. This is beyond the specificities of cell, organelle and transcriptional program targeting mentioned above. A core understanding of the method provided by this invention is that it achieves much higher relative concentrations of liganded immodulin peptide in the targeted peripheral immune tissues, as compared to the abdominal organs such as heart, liver, kidney, gastrointestinal tract, lung and pancreas in which drug toxicities are typically registered—or plasma, wherein concentrations of drug can serve as a surrogate for the exposure suffered by abdominal organs—such that the TI is seen to be expanded by favorable tissue distribution in the treatment of peripheral immune dysfunction. This is in addition to the other intracellular targeting advantages, which independently alter TI favorably by the specificity of intracellular targeting compared to traditional drug forms. Taken together, the features of immodulin-SMNRLs may offer the possibility of far favorable therapeutic indexes than are offered by current conventional treatments for peripheral immune dysfunctions.

Using amide bonds that resemble those naturally present in peptides and proteins for the covalent addition of small molecules to peptides is desirable. PCT/US2020/024828 teaches methods and compositions based on N-terminal covalent addition of non-amino acid carboxylate small molecule ligands to immodulin peptides, a conjugation step that occurs after assembly of the amino acid chain in synthesis. PCT/US2023/013908 teaches methods and compositions based on a technically-challenging C-terminal covalent addition of non-amino-acid carboxylate small-molecule ligands to immodulin peptides in a conjugation step that occurs prior to full amino acid chain elongation in synthesis. Each of these two approaches answers a different set of technical challenges that are industrially relevant to manufacture of immodulin-based targeted drugs at scale.

The present invention utilizes biodegradable polymers to enhance the transport and localization of liganded immodulins with greater efficiency and, if desired, with tunable release kinetics. Surprisingly, application of the formulations disclosed herein results in a unique biodistribution of the liganded immodulin in live rodents that predicts substantial improvements in therapeutic index—and, therefore, safety and efficacy—when targeting the peripheral immune tissues in the skin. Compared to traditional dosage forms, the relative concentration of agent in these tissues relative to plasma, or relative to the abdominal organs associated with most drug toxicities described in the scientific literature (liver, kidney, gastrointestinal tract, lung, heart and brain, for example) is significantly higher using the compositions and methods disclosed.

Recovery of therapeutics from widely employed biodegradable polymers such as poly-lactic-co-glycolic acid (PLGA) is often limited. This is thought to be a result of aggregation of the therapeutic molecule during the encapsulation process. Herein disclosed for the first time is the observation that mixing an immodulin with recombinant human holo-transferrin—which is readily available in GMP grade at reasonable cost from numerous suppliers—can improve the percentage of recovered therapeutic by more than three-fold. This is an example of the many practical uses of combined improvements described in this invention.

Rapid metabolism of therapeutics in blood when routes of delivery such as intravenous infusion or subcutaneous bolus injections are used in the administration of medicines is a serious, real-world complication in most drug-delivery contexts. Herein disclosed for the first time is the observation that mixing high-molecular-weight hyaluronic acid—which is readily available at reasonable cost at GMP grade from numerous suppliers—into the diluent used in the methods of this invention can improve the targeting to lymphatic tissues and result in greater efficacy for a given dose of modified immodulin. This is an example of the many practical uses of the improvements described in this invention.

The above are a few examples of the many aspects of this invention which, in the aggregate, provide significant real-world improvements to specificity, efficacy and safety in a whole class of therapeutic molecules built upon a ligand-modified immodulin peptide scaffold. The benefits achieved by the particular compositions and methods described herein are designed to have a combinatorial effect wherein the whole is greater than the parts. The many particular elements of the invention are either new when used alone or new when used in the combinations described.

Immodulin peptides are believed to rapidly select and enter target cells and travel to specific cellular compartments (e.g. the nucleus), where they interact with cellular machinery in epigenetic ways. For example, they may bind transcriptional machinery and alter large transcriptional sets. A comprehensive description of immodulins can be found in PCT/US2020/024828, PCT/US2021/021433, PCT/US2021/046814, PCT/US2022/018449 and in a recent publication (Mascarenhas, 2022) as well as in references cited therein.

Retinoid X receptors (RXRs) are promiscuous partners in heterodimeric associations with other members of the Nuclear Receptor (NR) superfamily. RXR ligands (“rexinoids”) may transcriptionally activate heterodimers (such as RXRγ/Nur77, or RXRα/PPARα). RXRs are obligatory partners for a number of other NRs, giving RXRs a coordinating role at the crossroads of multiple signaling pathways. RXRs represent important targets for pharmacologic interventions. Receptor knockout studies demonstrate an important role for these receptors both during development and in adult differentiated tissues (cell proliferation, cell differentiation, cell death). These receptors also play an important regulatory role metabolic signaling pathways (glucose, fatty acid and cholesterol metabolism), including metabolic disorders such as type 2 diabetes, hyperlipidemia and atherosclerosis. RXRs function as master regulators producing diverse physiological effects through the activation of multiple nuclear receptor complexes. The retinoid X receptor (RXR) subgroup (NR2B) of NRs is composed of 3 members: RXRα (NR2B1), RXRβ (NR2B2), and RXRγ (NR2B3). The transcriptional activity of RXR mainly results from its capacity to act as a cognate partner for other NRs. RXR can engage in 3 types of partnerships, permissive, conditional and non-permissive. Non-permissive heterodimers, such as RXR/VDR (vitamin D receptor) and RXR/TR (thyroid hormone receptor), are activated only by agonists of the partner. Conditional heterodimers, such as RXR/RAR (retinoic acid receptor), are not activated by RXR agonists, but the activity of agonists of the RXR partner receptor is enhanced by RXR agonists (synergistic effect). RXR agonists alone, partner receptor agonists alone or a combination of both can activate permissive heterodimers. Such complexes include heterodimers formed with PPAR (peroxisome proliferator-activated receptor), FXR (farnesoid X receptor), LXR (liver X receptor), and the orphan NR4A group NRs Nur77 and Nurr1. The NR4A subgroup of nuclear receptors includes Nur77 (NR4A1, also known as NGFI-B or TR3), Nurr1 (NR4A2) and Nor-1 (NR4A3). Nur77 and Nurr1 transcriptional activities can be indirectly manipulated through modulation of their heterodimeric partner RXR, using rexinoids such as LG100754, SR11237, BRF110, HX531, HX630 or HX600, or other RXR/NR4A ligands and modulators such as spironolactone, haloperidol, C-DIM12, C-DIM8 and cilostazol.

This invention discloses new compositions and improved utilities of the liganded-immodulin peptide class. Sequence extensions to the immodulin peptide sequences themselves can also confer extended biological activities useful in medicine and consumer products. Sequence extension of immodulin peptides has been described in PCT/US2020/024828, PCT/US2023/013908, PCT/US2022/018449 and PCT/US2021/046814, as well as in U.S. Pat. No. 5,519,003/5,783,405/6,165,977/6,262,023/6,342,368/6,423,684/6,855,693/6,933,275/7,393,835/8,536,135/10,369,1919 and references cited therein.

1. In one aspect, the invention provides a method for improving the therapeutic index of a small-molecule nuclear receptor ligand in the treatment of the peripheral immune tissues of a mammalian subject comprising:
(a) chemical synthesis of a peptide 20-60 amino acids long comprising an amino acid sequence corresponding to SEQ ID NO:1 or SEQ ID NO:2;
(b) covalent attachment of the small-molecule nuclear receptor ligand to the synthetic peptide; (c) purification of the ligand-modified peptide;
(d) mixing of the purified ligand-modified peptide with purified recombinant human transferrin at a peptide-to-transferrin molar ratio of between 0.01 and 10;
(e) trapping of the peptide-transferrin mixture in a biodegradable polymer formulation;
(f) suspension of the formulation in a suitable pharmaceutical diluent containing at least 0.001% high-molecular-weight hyaluronic acid;
(g) administration of the formulation to a mammalian subject via a transdermal or intradermal route; thereby achieving at least a two-fold expansion of therapeutic index of the small-molecule nuclear receptor ligand-modified peptide in the treatment of the peripheral immune system when compared to a molar equivalent dose of the same small-molecule nuclear receptor ligand administered alone by the same route.

In some embodiments the invention provides a liganded immodulin peptide wherein the small-molecule nuclear receptor ligand of mass less than about 1000 daltons is selected from a group consisting of HX600, HX630, celastrol, LG100754, GW7647, tamibarotene, GW3965, AM580, palovarotene, adapalene, bexarotene, capric acid, fenofibric acid, GW4064, sobetirol and GW501516.

In some embodiments, the tissue collected from the mammalian subject is selected from a group consisting of plasma, serum, blood cells, liver, kidney, lung, heart, gastrointestinal tract, pancreas, upper limb skin, lower limb skin, neck skin, cervical lymph node, popliteal lymph node, axillary lymph node, brachial lymph node and epitrochlear lymph node.

In some embodiments, the diluent used in the administration of the liganded peptide contains at least 0.001% of high-molecular-weight hyaluronic acid and/or a botanical compound selected from a group consisting of resveratrol, quercetin, menthol, anisic acid, verbenone, eugenol, linalool, ferulic acid, glycyrrhizic acid, levulinic acid, alkali salt of olive oil and alkali salt of coconut oil.

In some embodiments the liganded peptide is complexed with recombinant human holo-transferrin and encapsulated in a biodegradable polymer such as poly-lactic-co-glycolic acid or polycaprolactone.

In yet another related aspect, the invention provides a method for treating a subject suffering from immunological, neurological, oncologic, skeletal, reproductive, metabolic or cosmetic dysfunction or perturbation, where the method includes administering to the subject (e.g., a human subject) a therapeutically effective dose of liganded peptide, peptide complex, or other pharmaceutical composition described in the invention. In some embodiments the therapeutically effective dose of the liganded immodulin peptide is from about 0.01 mg/kg/day to about 50 mg/kg/day.

In yet another related aspect, the invention provides an in vitro method for measuring the potency of any immodulin peptide described herein using cultured mammalian cells.

### DEFINITIONS AND DETAILED DESCRIPTION

“Therapeutic index” (TI) is a ratio that expresses the relationship between the dose expected to elicit some adverse effect (e.g., LD₅₀, TD₅₀, etc.) and the dose needed to elicit therapeutic effects (e.g., ED₅₀).

The term “functional therapeutic index” shall mean the actual therapeutic index achieved in vivo by a combination of drug design, formulation employed and anatomical route of administration.

Functional therapeutic index can be calculated from dose-elicited drug concentrations in plasma (as measured by Cmax or AUC) which are known to elicit adverse effects (e.g., LD₅₀, TD₅₀, etc.) in major abdominal organs such as liver, kidney, heart, gastrointestinal tract, lung or pancreas—versus dose-elicited drug concentrations needed for therapeutic effects (e.g., ED₅₀) in target tissues, such as peripheral immune tissues (PIT).

The terms “subject” and “individual”, as used herein, refer to mammalian individuals, and more particularly to domesticated animals (e.g., dogs, cats, and the like), agricultural animals (e.g., cows, horses, sheep, and the like), and primates (e.g., humans).

The terms “administration” or “treatment” are used herein are precursors to the term “alleviating”, which, as used herein, refers to an improvement, lessening, stabilization, or diminution of a symptom of a disease or immune perturbation. “Alleviating” also includes slowing or halting progression of a symptom.

The term “liganded immodulin peptide” shall mean a peptide molecule 20-60 amino acids in length prepared by chemical synthesis and comprising an immodulin peptide sequence corresponding to SEQ ID NO:1 or SEQ ID NO:2, modified by conjugation to at least one small bioactive molecule that is not an amino acid, and optionally further modified by one or more of the following modifications: (a) extension with a non-IGFBP sequence; (b) stabilization using D-isomers; (c) complexation to a metal, transferrin, glycosaminoglycan or helper molecule; or (d) formulation as nanoparticle or microparticle, optionally coated with a chitosan compound and/or human serum albumin. Human serum albumin used herein may additionally be complexed to a homing peptide.

“RXR” means retinoid X receptor, and can refer to either the RXR gene or the protein it specifies. “Rexinoid” means a ligand of an RXR receptor. “RXRs” means any of the RXR isoforms, such as RXR-alpha (RXRα), RXR-beta (RXRβ), RXR-gamma (RXRγ), which may form heterodimers with other nuclear receptors (NRs) in their own class, or in other classes.

RXRs are known to form heterodimers with members of several classes of NRs. NRs are classified into seven classes, NR0 through NR6. It is known that classes NR1-NR4 contain heterodimeric partners of RXRs. Each class contains subclasses e.g. class NR1 contains NR1A, NR1B, NR1C, NR1D, NR1F, NR1H and NR11. Members of at least 5 of these subclasses can form heterodimers with RXRs. Members of NR2, NR3 and NR4 classes also contain members capable of forming heterodimers with RXRs (NR2B class contains the RXRs themselves, and they can homodimerize). A note about nomenclature: The term “NR4A class” includes the orphan nuclear receptors NR4A1, NR4A2 and NR4A3, each one of which can heterodimerize with RXRs. NR4A1 can refer to either the NR4A1 gene or the protein it specifies. The protein, in turn, may have one or more common names in the literature (in the case of NR4A1, the names Nur77, TR3 or NGF1-B). RXR receptors can form functional heterodimers with efficiencies that vary with RXR isoform, actual tissue distribution of each nuclear receptor under a given biological context, epigenetic modulators, and many other variables.

Nuclear receptor classes 1 through 4 contain the following members:

| Class | Protein |
|---|---|
| NR1A1 | TRα |
| NR1A2 | TRβ |
| NR1B1 | RARα |
| NR1B2 | RARβ |
| NR1B3 | RARγ |
| NR1C1 | PPARα |
| NR1C2 | PPARβ/δ |
| NR1C3 | PPARγ |
| NR1D1 | REV-ERBα |
| NR1D2 | REV-ERBβ |
| NR1F1 | RORα |
| NR1F2 | RORβ |
| NR1F3 | RORγ |
| NR1H2 | LXRβ |
| NR1H3 | LXRα |
| NR1H4 | FXRα |
| NR1I1 | VDR |
| NR1I2 | PXR |
| NR1I3 | CAR |
| NR2A1 | HNF4α |
| NR2A2 | HNF4γ |
| NR2B1 | RXRα |
| NR2B2 | RXRβ |
| NR2B3 | RXRγ |
| NR2C1 | TR2 |
| NR2C2 | TR4 |
| NR2E1 | TLX |
| NR2E3 | PNR |
| NR2F1 | COUP-TFα |
| NR2F2 | COUP-TFβ |
| NR2F6 | COUP-TFγ |
| NR3A1 | ERα |
| NR3A2 | ERβ |
| NR3B1 | ERRα |
| NR3B2 | ERRβ |
| NR3B3 | ERRγ |
| NR3C2 | MR |
| NR3C3 | PR |
| NR3CI | GR |
| NR3I4 | AR |
| NR4A1 | Nur77 |
| NR4A2 | NURR1 |
| NR4A3 | NOR-1 |

This invention envisages an in vitro assay method for measuring the biological potency or biodistribution of an immodulin peptide. As will be understood by those of skill in the art, the mode of detection of a diagnostic signal will depend on the exact detection system utilized in the assay. For example, if a fluorescent detection reagent is utilized, the signal will be measured using a technology capable of quantitating the signal from the sample, such as by the use of a fluorometer. If a chemiluminescent detection system is used, then the signal will typically be detected using a luminometer. Methods for assaying fluorescent or biological signals are well known in the art.

Sequence “identity” and “homology”, as referred to herein, can be determined using BLAST, particularly BLASTp as implemented by the National Center for Biotechnology Information (NCBI), using default parameters. It will be readily apparent to a practitioner skilled in the art that sequences claimed hereunder include all homologous and trivial variants of an immodulin peptide, such as by conservative substitution, extension and deletion in their amino acid sequences. Trivial substitution variants include swapping of an amino acid with another belonging to the same class, without such substitution resulting in any significant and measurable functional improvement. “Classes” of amino acids include positively charged amino acids (arginine, lysine, histidine), negatively charged amino acids (aspartic acid, glutamic acid), aromatic amino acids (tyrosine, phenylalanine, tryptophan), branched chain amino acids (valine, leucine isoleucine) and other natural groupings such as (serine, threonine) and (asparagine, glutamine).

As will be understood by those of skill in the art, the symptoms of disease alleviated by the instant methods, as well as the methods used to measure the symptom(s) will vary, depending on the particular disease and the individual patient. All references cited in this document, including patent applications and publications cited therein, are incorporated by reference in their entirety.

### EXAMPLES

Example 1. C-terminal modification of peptide with non-amino acid carboxylic acids of low molecular mass. N-terminal modification of peptides with biotin, or fatty acids such as myristic and palmitic acids, is widely known in the field. Other types of carboxylic acids have rarely been used in this way, largely due to technical difficulties in recovering the correct product. For immodulin peptides, previous disclosures of N-terminal modification using non-conventional compounds include PCT/US2022/018449, PCT/US2021/046814, PCT/US2021/021433 and PCT/US2020/024828, as well as scientific publications (see, for example, Mascarenhas, 2022 and references cited therein). PCT/US2020/024828 teaches methods and compositions based on N-terminal covalent addition of non-amino acid carboxylate small molecules to immodulin peptides, a conjugation step that occurs after assembly of the amino acid part of the chain in synthesis. Sufficient molar ratios of the non-amino acid carboxylate must be used in that process, in order to satisfy not just a reaction with the correctly assembled chain, but also the much more numerous failure sequences (such as deletions) accumulated during chain elongation. For immodulin peptides, which can often be between 35 and 45 amino acid residues in length (and sometimes longer), such failure sequences can significantly raise the cost of identification and recovery of useful product from the reaction mixture. In practice, therefore, the use of the N-terminal addition method disclosed in PCT/US2020/024828 is largely limited by the cost of the said non-amino acid carboxylate reagent. the covalent addition of non-amino acid carboxylates to a C-terminally located lysine in the immodulin peptide sequence. This reaction occurs, near the start of amino acid chain elongation during synthesis. Regardless of the length of the final peptide chain, the option to couple a non-amino acid carboxylate to the epsilon amino group of a C-terminal lysine monomer via a covalent bond obviates the abovementioned technical problems encountered with the method disclosed in PCT/US2020/024828, vastly increasing the options previously foreclosed due to cost. In short, C-terminal addition allows for the addition of more expensive compounds. As described, the invention provides for the addition of non-amino acid carboxylates wherein said molecules can bind a nuclear receptor with sub-millimolar affinity in vitro. There are, however, novel technical issues that limit C-terminal in-synthesis conjugation of such non-amino acid carboxylates. For example, said compounds must be stable enough to survive multiple cycles of synthesis under the harsh conditions typically employed for automated peptide synthesis. For some compounds, if the conjugation is not efficient, the C-terminal monomer may need to be modified and pre-purified prior to use in chain elongation. The general methods disclosed herein are applicable to a number of compounds that are capable of binding a nuclear receptor with sub-millimolar affinity in vitro. In practice, however, it is not always possible to predict in advance which ligands will work satisfactorily, and which will not. None of the nuclear-receptor-binding compounds successfully exemplified here have ever been previously coupled to the C-terminal lysine residue of an immodulin peptide (or, indeed, to the best of the inventor's knowledge, to any other peptide). This invention discloses, for the first time, small non-amino acid carboxylates that can bind a nuclear receptor with sub-millimolar affinity in vitro conjugated to the C-terminus of an immodulin peptide during synthesis. The peptides were synthesized on ChemMatrix Rink Amide resin, as follows, using a modified Fmoc synthesis protocol with DIC/CI-HOBt coupling, on an APEX 396 automatic synthesizer. For example, using GW7647 as the compound, Fmoc-d-Lys(GW7647)-OH was customized in this way: GW7647 was activated as an NHS-ester and then reacted with Fmoc-d-Lys-OH, which had a free epsilon amine on its side chain. The FMOC-d-Lys(GW7647) wang resin was swollen in dimethylformamide (DMF) for 30 min, treated with 20v % Piperidine-DMF for 8 minutes to remove the Fmoc protecting group at 50° C., and washed with DMF three times. For the coupling reaction, the resin was added with Fmoc-protected amino acid, CI-HOBt, DIC and NMP. The mixture was vortexed for 20 minutes at 50° C. Afterwards, the resin was washed with DMF once. The cycle of deprotection and coupling steps was repeated until the last biotin residue was assembled. The resin was then washed with DMF, DCM and dried with air. The peptide was cleaved from the resin using a TFA cocktail (95v % TFA, 2.5v % water and 2.5v % TIS) for three hours. Crude peptides were precipitated by adding ice-chilled anhydrous ethyl ether, washed with anhydrous ethyl ether for three times, and dried in vacuo. The purification was performed accordingly by a prep-HPLC. The results of the conjugation experiments show that there is wide variation in conjugation efficiency from compound to compound. As the practicality and cost of synthesis can be dramatically affected when product yield is low, it is therefore not obvious a prior that any untested carboxylic acid should be assumed to be a good candidate for this type of peptide modification. The use of compounds tested here and listed in the table below has never been reported for this kind of peptide modification. It appears that that chance of practical success is low until shown. Once shown, however, the C-terminal modification of an immodulin peptide with a given compound is nearly always highly reproducible. Some examples are shown below:

TABLE X1.1
Small-molecule nuclear receptor ligands.

| Ligand | | | |
|---|---|---|---|
| Class | Compound | CAS No. | MW |
| PPARα | GW7647 | 265129-71-3 | 502.8 |
| PPARα | Fenofibric acid | 42017-89-0 | 318.8 |
| PPARd | GW501516 | 317318-70-0 | 453.5 |
| PPARγ | Capric acid (decanoate) | 1002-62-6 | 194.2 |
| FXR | GW4064 | 278779-30-9 | 542.8 |
| LXR | GW3965 | 405911-17-3 | 618.5 |
| RARβ/γ | Adapalene | 106685-40-9 | 412.5 |
| RARα/β | Tamibarotene (AM80) | 94497-51-5 | 351.4 |
| RARα | AM580 | 102121-60-8 | 351.4 |
| RARβ | BMS453 | 166977-43-1 | 380.5 |
| RARγ | Palovarotene | 410528-02-8 | 414.5 |
| RXR | Bexarotene | 153559-49-0 | 348.5 |
| RXR | Celastrol | 34157-83-0 | 450.6 |
| RXR | HX600 | 172705-89-4 | 438.6 |
| RXR | LG100754 | 180713-37-5 | 396.6 |

TABLE X1.2
Examples of C-terminally liganded immodulin peptides

| Peptide ID | Peptide sequence | Ligand moiety | Ligand for |
|---|---|---|---|
| imm3ppa | SDKKGFYKKKQCRPSKGRKRGFCWSVDK | GW7647 | PPARα |
| 3*GW9 | KKGFYKKKQCRPSKGRKRGFCWAVDK | GW3965 | LXR |
| imm3dec | SDKKGFYKKKQCRPSKGRKRGFCWSVDK | Capric | PPARγ |
| imm3fxr | SDKKGFYKKKQCRPSKGRKRGFCWSVDK | GW4064 | FXR |
| imm3adp | KKGFYKKKQCRPSKGRKRGFCWSVDK | adapalene | RARß/γ |
| imm3tam | SDKKGFYKKKQCRPSKGRKRGFCWAVDK | tamibarotene | RARα/ß |
| imm3rag | SDKKGFYKKKQCRPSKGRKRGFCWSVDK | palovarotene | RARγ |
| imm3raa | SDKKGFYKKKQCRPSKGRKRGFCWSVDK | AM580 | RARα |
| imm3bex | SDKKGFYKKKQCRPSKGRKRGFCWSVDK | bexarotene | RXR |
| 3*cel | KKGFYKKKQCRPSKGRKRGFCWAVDK | celastrol | RXR |
| 3*LG7 | KKGFYKKKQCRPSKGRKRGFCWAVDK | LG100754 | RXR |
| imm3K1tam | SLNPEWNETKKGFYKKKQCRPSKGRKRGFCWAVDK | tamibarotene | RARα/ß |
| imm3K1 | SLNPEWNETKKGFYKKKQCRPSKGRKRGFCWAVD | | control |
| imm3AVD | KKQCRPSKGRKRGFCWAVD | | control |
| Imm3SVD | KKQCRPSKGRKRGFCWSVD | | control |

Practitioners well versed in the art will recognize the possibility of additionally adding NR ligands to the N-terminus of a carboxyterminally liganded-immodulin, using the methods for N-terminal modification referenced above and in the examples below. For example, ligand GW4064 can be added to the N-terminus of liganded-immodulin imm3bex, to create imm3fxr-bex: [fxr-SDKKGFYKKKQCRPSKGRKRGFCWSVDK-[bex]. This type of construction allows two different ligands to modify transcription within the same cell nucleus simultaneously. Adding the two ligands separately to achieve an identical dual-transcriptional result in a live mammal would not be possible because of dose-limited toxicities. No peptide carrying two different classes of NR ligand to the nucleus simultaneously has ever been described. Liganded-immodulins thus pave the way for novel and complex interventions in medicine.

Biological effects of liganded immodulins can be shown in many different biological systems, both in vitro and in vivo. Mascarenhas (2022) provides detailed descriptions of assays for CD169+ macrophage generation and 02012 myoblast differentiation, both naturally mediated by immodulin peptides. Examples of the performance of liganded-immodulins in similar assays are shown in the tables below. Table X1.3 shows examples of CID169+ macrophage differentiation and subsequent effects on T-helper (Th) subsets by co-culture with CD4+naïve T-helper cells. Briefly, macrophage differentiation and polarization assays were done using the THP1-Dual monocyte reporter cell line (Invivogen Inc, San Diego, CA) seeded at 2×10e5 cells per well in 96-well plates and cultured at 37 degrees C. in RPMI-1640 growth medium plus 10% fetal bovine serum and 1% penicillin/streptomycin, then treated for 24 hours with 100 ng/ml Phorbol 12-myristate 13-acetate (macrophage PMA protocol). Peptide (330 or 660 nM) was then added, and incubation continued for an additional 24 hours. Culture supernatants were then assayed for C—C motif chemokine 22 (CCL22), interleukin-10 (IL-10) or transforming growth factor beta (TGFbeta) using Duoset ELISA kits (R&D Systems, Minneapolis, MN). Adherent cells were washed twice with PBS and cells were assayed for immunoreactivity of surface markers CD169, Clec9a and Clecl2a using biotin-labeled anti-human antibodies for these markers purchased from Miltenyi Biotec (Auburn, CA) and a streptavidin-horseradish peroxidase/TMB secondary detection reagent. Results were expressed as arbitrary ELISA immunoreactivity units relative to the control peptide (=100). For T-cell differentiation co-culture assays, the THP1-Dual monocyte reporter cell line was treated with PMA for 24 hours as described above, adherent cells were washed twice with PBS and approximately 2×10e5 naïve CD4+ T-cells (Zen-Bio Inc, Durham, NC) were layered on the macrophages in 100 uL RPMI-1640 containing peptide and 15 uL T-activator CD3/CD28 Dynabeads (Thermofisher Scientific, Waltham, MA) and incubated for a further 96 hours. Protein was assayed using the BCA protein assay kit from Thermofisher Scientific (Waltham, MA). AU=arbitrary units (immunoreactivity) per milligram protein: Control 100=peptide imm3K1tam.

| | Peptide (0.66 uM)> | | | |
|---|---|---|---|---|
| | imm3K1tam | imm3K1 | imm3AVD | Buffer |
| CD169 (AU) | 100 ± 4.7 | 24.3 ± 1.6** | 22.1 ± 3.2** | 3.1 ± 2.7** |
| CCL22 (ng/ml) | 6.2 ± 0.3 | 1.9 ± 0.1** | 0.6 ± 0.1** | 0.2 ± 0.1** |
| CXCL8 (ng/ml) | 8.9 ± 0.3 | 2.7 ± 0.2** | n.d. | 0.1 ± 0.0** |
| IL-10 (ng/ml) | 17.2 ± 0.7 | 4.7 ± 0.3** | 1.3 ± 0.1** | 0.1 ± 0.1** |
| TGF-beta (ng/ml) | Not done | 0.8 ± 0.1# | 0.1 ± 0.0 | 0.0 ± 0.0 |
| Treg (FoxP3 AU) | 100 ± 3.5 | 85.4 ± 2.6** | 46.5 ± 8.5** | 31.6 ± 5.6** |
| Th17 (IL-17 pg/ml) | 35 ± 7 | 235 ± 33** | Not done | 125 ± 27** |
| Th2 (IL-13 AU) | 100 ± 12 | 120 ± 48 | Not done | 120 ± 16 |

Table X1.3 shows examples of CD169+ macrophage deffertiation (and subsequent effects of Th subsets by co-culture with CD4+ native T-helper cells).
**p < 0.01
*p < 0.05 vs imm3K1tam control;
#p < 0.01 vs imm3AVD control.

Table X1.4 shows data from C2C12 myoblast differentiation. C2C12 myoblast cell line (ATCC, Manassas, VA) was cultured in DMEM medium supplemented with 10% FBS and 1% penicillin/streptomycin. For differentiation, cells were cultured in the same medium, except that 10% FBS was replaced by 2% horse serum. Peptides and NR ligands were added at the indicated concentrations and cells were harvested 96 hours later for assay. Cell extracts were assayed for creatine kinase (CK) per mg protein. CK was measured using the ECPK-100 kit from BioAssay Systems (Hayward, CA). Protein was assayed using the BCA protein assay kit from Thermofisher Scientific (Waltham, MA). CK activities and p values were expressed relative to imm3SVD peptide (=100). **p<0.01; *p<00.05.

TABLE X1.4
Data from C2C12 myoblast differentiation assay.

| Peptide | | | |
|---|---|---|---|
| (0.66 uM) | Added ligand (2 uM) | Agonist of | CK (units/mg) |
| None | None | None | 5.2 ± 2.1** |
| imm3SVD | None | None | 100.0 ± 2.9 |
| imm3SVD | GW7647 | PPARα | 163.3 ± 2.0** |
| imm3SVD | GW9578 | PPARα | 185.6 ± 10.5** |
| imm3SVD | Ciprofibrate | PPARα | 109.4 ± 3.2* |
| imm3SVD | GW501516 | PPARδ | 174.2 ± 5.0** |
| imm3SVD | GW3965 | LXR | 67.4 ± 5.2** |
| imm3SVD | Capric acid | PPARγ | 116.1 ± 23.2 |
| imm3SVD | GW4064 | FXR | 165.5 ± 11.9** |
| imm3SVD | tamibarotene | RARα/β | 88.0 ± 32.7 |
| imm3SVD | sobetirome | THRβ | 220.7 ± 11.9** |
| imm3SVD | bexarotene | RXR | 124.8 ± 13.2* |
| imm3SVD | HX531 | RXR | 41.1 ± 9.9** |
| imm3SVD | HX600 | RXR | 30.3 ± 16.0* |
| imm3SVD | LG100754 | RXR | 193.9 ± 30.8** |
| imm3bex | bexarotene (C-liganded) | RXR | 121.0 ± 6.6* |
| imm3fxr | GW4064 (C-liganded) | FXR | 136.8 ± 19.6* |
| imm3ppa | GW7646 (C-liganded) | PPARα | 155.9 ± 18.8** |
| imm3adp | adapalene (C-liganded) | RARβ/γ | 185.8 ± 23.5** |
| imm3bex + | | RXR + | 221.3 ± 34.4** |
| imm3fxr | | FXR | |
| imm3bex + | | RXR + | 236.8 ± 29.2** |
| imm3ppa | | PPARα | |

By addition of an additional NR ligand to the C-terminus of an liganded-immodulin using the methods described in Examples 1 and 2 above, it is possible to combine the effects of multiple transcriptional pathways at will. For example, the effects of imm3fxr and imm3bex were combined into a single peptide imm3fxr-bex, wherein GW4064 and bexarotene were added to the N— and C— terminii, respectively, of a peptide of identical sequence, In cell culture assays, imm3fxr-bex performs as predicted from the performance of the combination of imm3fxr and imm3bex liganded-immodulins, an example of which is shown in the table above. As noted previously, however, the in vivo performance of imm3fxr-bex (and other such single-molecule combinations, hereinafter termed “multisynchrons”) is expected to be technically and industrially superior, owing to dose-related and regulatory limitations associated with such combinations.

Example 2. N-terminal modification of peptide with non-amino acid carboxylic acids of low molecular mass. N-terminal modification of peptides with biotin, or fatty acids such as myristic and palmitic acids, has been widely used. This example shows the difficulty in predicting success for this type of modification for other carboxylic acids. The data in this example disclose, amongst other facts: (i) coupling to carboxylic acids (that are not proteinogenic amino acids or biotin) using normal peptide synthesis conditions; (ii) surprising results showing that yields of correctly coupled product (as ascertained by liquid chromatography and mass spectroscopy analysis) varied greatly, even within the same class of compound; and (iii) consistent results for small molecules attached by this method to immodulin peptide or a generic D-tetrapeptide dLys-dAsp-dLys-dPro, with similar efficiencies of coupling to either peptide, thereby demonstrating the generality of the method. Peptides were synthesized according to a common Fmoc/tBu solid phase synthesis strategy well-known in the art. Synthesis may be manual of automated. After the peptide synthesis the resin was divided into batches of 20 umol. Each batch was treated with one of the organic compounds specified in Table X2.1 below. The coupling was carried out using 2 equivalents of the compound, 2.4 equivalents of activator HATU or HCTU, and 4 equivalents of NMM base. The reaction mixture was renewed after 2 hrs reaction time and allowed to react another 4 hrs or overnight. After washing the resin several times with DMF, and subsequently with DCM, the batches were dried. For the cleavage of the peptides from the resin the resins were treated with 1% DTT, 2% water and 3% TIPS in TEA for 3.5 hrs. The cleavage solution was separated from the resin and treated with diethylether/n-pentane (1:1). The resulting precipitate was centrifuged and the pellet washed three times in the same DEE/pentane mixture. The recovered peptide was air dried and stored at −20 degrees C. or further purified by HPLC using a 0-50% acetonitrile gradient, 0.1% trifuoroacetic acid (20 m). The results of the above conjugation experiments show that, both inter-class and intra-class, there is wide variation in conjugation efficiency from compound to compound. It is therefore not obvious if any untested carboxylic acid will be a good candidate for this type of peptide modification. The use of most of the compounds tested here has never been reported for this kind of peptide modification. It appears that establishing utility (>80% correct yield, for instance) requires prior testing.

TABLE X2.1
N-terminal coupling of non-amino acid carboxylates to immodulin peptides.

| Class | Compound | CAS No. | MW | Yld T4* | Yld IM3* |
|---|---|---|---|---|---|
| fatty acid | oleic acid | 112-80-1 | 282.5 | 44.21% | |
| fatty acid | eicosapentaenoic acid | 10417-94-4 | 302.5 | 66.79% | |
| fatty acid | lignoceric acid | 557-59-5 | 368.6 | 89.20% | |
| fatty acid | capric acid | 1002-62-6 | 172.2 | 88.67% | 98.0% |
| fatty acid | docosahexanoic acid | 6217-54-5 | 368.6 | 57.77% | |
| fatty acid | lauric acid | 143-07-7 | 200.3 | 85.14% | 96.7% |
| fatty acid | 10-hydroxy-2-decenoic acid | 14113-05-4 | 186.3 | 44.38% | |
| phenolic acid | ferulic acid | 1135-24-6 | 194.2 | 26.58% | |
| phenolic acid | isoferulic acid | 537-73-5 | 194.2 | 55.80% | 70.2% |
| phenolic acid | Aspirin | 50-78-2 | 180.2 | 56.5% | |
| phenolic acid | valeroyl salicylate | 64206-54-8 | 222.2 | 11.76% | |
| pentacyclic | betulinic acid | 472-15-1 | 456.7 | <1% | |
| anthraquinone | Rhein | 478-43-3 | 284.2 | 50.95% | |
| anthraquinone | Diacerein | 13939-02-1 | 368.3 | 43.2% | |
| Xanthone | 2,7-dichlorodihydro- | 4091-99-0 | 487.3 | 91.2% | |
| proprionic acid | (s)-ketoprofen | 22161-81-5 | 254.3 | 77.86% | |
| proprionic acid | Ibuprofen | 15687-27-1 | 206.3 | 93.42% | 98.0% |
| carboxylic acid | trans-cinnamic acid | 140-10-3 | 148.2 | 93.12% | 81.5% |
| carboxylic acid | (s)-(−)-perillic acid | 23635-14-5 | 166.2 | 27.96% | |
| carboxylic acid | fenofibric acid | 42017-89-0 | 318.8 | 85.67% | 99.9% |
| indoleacetic acid | Indomethacin | 53-86-1 | 357.8 | 87.5% | 85.2%# |
| pentanoic acid | valproic acid | 1069-66-5 | 144.2 | 91.43% | 84.9% |
| alkynoic acid | 2-hexyl-pentynoic acid | 96017-59-3 | 182.3 | | 85.1% |
| indolylcarboxylic | RG-108 | 48208-26-0 | 334.3 | | 74.3%@ |
| Retinoid | all-trans retinoic acid | 302-79-4 | 300.4 | 13.1% | |
| Rexinoid | Bexarotene | 153559-49-0 | 348.5 | 97.09% | 94.4% |

Covalent terminal conjugation of carboxylic acids to peptides.
*Percent yield of correct species by MS for T4 (tetrapeptide) and IM3 immodulin peptide (industrially acceptable >80% shown in bold type);
#lost p-chlorophenone group (incorrect product);
@indole core oxidized by Arg protecting groups (incorrect product).

Example 3. Biodistribution of immodulins in vivo. In the ideal scenario, drug accumulates at therapeutic levels at a desired tissue location in the body while simultaneously exhibiting low levels in plasma. Therapeutic peptides can be encapsulated in PLGA microparticles or nanoparticles by a number of protocols widely known in the art. For encapsulation in microparticles containing immodulin peptides, the following protocol can be used: All solutions are filter-sterile. PLGA-M solution contains 50 mg/ml poly-lactide-co-glycolide (acid terminated) i.e. PLGA-COOH (Cat. #26268-1, Polysciences Inc, Warrington, PA) dissolved in acetone containing 4 mg/ml menthol. For example, peptides imm3K1tam or imm3SVD can be used. Microparticles are assembled as follows: 50 uL (100 ug) fluorescent-tagged-peptide (or HRP enzyme-tagged-peptide) solution is added to 75 uL saline buffer (FITC) or 75 uL recombinant human holo-transferrin (FITC-Tf). 1 ml PLGA-M solution is then added and the mixture is vortexed thoroughly. 2 ml of 2% polyvinyl alcohol is added, followed by vortexing. The resulting mixture is then added to 50 ml tubes containing 20 ml sterile distilled water and vortexed again. The tube is shaken at room temperature with the cap off for 2-4 hours to allow evaporation of the solvent. Particles are collected by centrifugation at 4,000 rpm in a Beckman centrifuge for 10 minutes. Particles may optionally be coated by resuspension in 500 uL of 2 mg/ml glycol chitosan (or other chitosan compound) for 30 mins at RT, then 500 uL of 10 mg/ml human serum albumin (HSA) is added and incubation at RT continued for 30 mins, followed by centrifugation. (HSA may be pre-conjugated to a homing peptide. Homing peptides have been described in the scientific literature for decades and are well known in the art.) Finally, the particles are washed 3 times with 1 ml sterile distilled water and collected by centrifugation. In order to deliver the microparticles, they may be suspended in 10×NPDF nanoemulsion for transdermal delivery, 1×NPDF in saline for intranasal delivery, or saline for sub-cutaneous bolus delivery. 10×NPDF is prepared as follows: Base solvent is made by mixing 2 ml sterile distilled water, 4 ml propylene glycol, 16 ml diethylene glycol monoethyl ether and 16 ml glycerol. The following are dissolved in base solvent: 5 mM each menthol, p-anisic acid and verbenone; 1 mM each linalool, ferulic acid and glycyrrhizic acid. Finally, 25 ml of this solution in base solvent is added to 25 ml of 6 mM sodium acetate pH 5.2 containing 0.9% sodium chloride, and 10 mM levulinic acid. By using immodulin peptides with or without PLGA encapsulation, significantly increased delivery to tissues using microparticles suspended in the above diluents has been demonstrated. Addition of hyaluronic acid to the diluent increases delivery to target tissues. Biodistribution of 50 uL each prep in rats (n=4 to 8 per group) is scored as fluorescent counts (or enzymatic units) per milligram tissue protein. Using this metric, the following results were obtained at 2 hours post-administration (normalized to plasma=1.0). Legend: Naked (SQn) and PLGA-encapsulated (SQ) imm3SVD peptide in 50 uL PBS buffer via subcutaneous bolus; IN=PLGA-encapsulated imm3SVD peptide in 50 uL 1×NPDF-saline; TDc (calf) or TDf (forearm) PLGA-encapsulated HRP-labeled imm3K1tam peptide in 50 uL 10×NPDF applied transdermally. All values shown are relative to the internal plasma control values i.e. showing relative biodistribution. Each tissue group is the average of five representative tissues in each group (see legend below table for details):

TABLE X3.1
Biodistribution of labeled immodulin peptides administered to rats via various routes.

| Admin Route | SQn ^{#} | SQ ^{#} | IN ^{#} | SQ ^{##} | TDc ^{##} | TDf ^{##} |
|---|---|---|---|---|---|---|
| Plasma | 1.00 ± 0.65 | 1.00 ± 0.05 | 1.00 ± 0.35 | 1.01 ± 0.00 | 1.00 ± 0.07 | 1.00 ± 0.23 |
| Peripheral | 1.91 ± 1.60 | 4.28 ± 0.86 | 3.95 ± 0.52 | 3.08 ± 0.75 | 10.12 ± 3.32 | 10.11 ± 1.55 |
| Immune | | | | | | |
| Group | | | | | | |
| Muscle | 2.05 ± 0.31 | 2.83 ± 0.46* | 1.83 ± 0.55** | 2.62 ± 0.77 | 1.34 ± 0.13* | 2.61 ± 0.44** |
| Group | | | | | | |
| Splanchnic | 2.15 ± 0.35 | 7.39 ± 3.82 | 6.67 ± 1.31** | 9.50 ± 4.42* | 2.83 ± 1.33* | 4.21 ± 1.61** |
| Group | | | | | | |
| T.I. ratio ^{&} | 0.61 | 1.04 | 1.42 | 1.00 | 6.40 | 3.30 |
| T.I ratio ^{&&} | 1.53 | 1.00 | 1.02 | 1.00 | 11.05 | 7.42 |

^{#} FITC label;
^{##} HRP label;
*p < 0.05
**p < 0.01 versus peripheral immune group;
Peripheral Immune Group (average): forlimb skin, back skin, calf skin, brachial lymph node, popliteal lymph node;
Muscle Group (average): biceps, triceps, calf EDL, soleus, flexor ulnaris;
Splanchnic Group (average): Small Intestine, kidney, heart, liver, pancreas);
T.I ratio:
^{&} = peripheral immune versus muscle group;
^{&&} = peripheral immune versus splanchnic group;
SQ = 1;
The results show a dramatic relative predicted expansion of functional T.I. when immodulin peptides are administered transdermally to upper or lower limb. In both cases, concentrations of peptide are enriched in both upper and lower limb peripheral lymphatic tissues (skin and surface draining lymph nodes), without corresponding increases in the plasma levels or in deep mesenteric lymph nodes (latter control was done, but is not shown). These results, taken together, suggest an active biological partitioning of immodulins to the peripheral lymphatic circulation.

Example 4. Improved release of immodulins from microparticles using transferrin. Functional TI can be further improved by using pharmaceutically acceptable formulations. Encapsulation of drugs in biodegradable PLGA microparticles is a widely employed technique in FDA-approved therapeutics, typically enhancing delivery characteristics of a drug. Actual use of PLGA encapsulation can be hampered by poor release of trapped drug. Surprisingly, co-encapsulation of immodulin-based drugs with recombinant human holo-transferrin results in a striking improvement in the release of immodulin peptides from PLGA particles. The use of transferrin to facilitate release of a peptide drug from PLGA particles is new. PLGA microparticles were prepared as described in Example 3. FITC-labeled peptide (FAM) was packaged either with or without transferrin, as described in Example 3. Release in NPDF diluent was measured by counting fluorescence in supernatants at 488 nm. Added transferrin improves release kinetics from microparticles by 300-400%.

TABLE X4.1
FITC-labeled immodulin peptide (FAM) with or without transferrin (Tf): Percent
released from PLGA microparticles in NPDF at 37 degrees C. over time.

| Hours >> | 0.0 | 0.4 | 1.4 | 3.4 | 8.1 | 14.5 | 18.5 |
|---|---|---|---|---|---|---|---|
| FAM | 2.3 ± 1.7 | 3.5 ± 0.9 | 5.7 ± 2.8 | 6.2 ± 1.3 | 8.8 ± 1.0 | 11.2 ± 1.4 | 13.9 ± 0.9 |
| FAM + Tf | 25.1 ± 1.1* | 22.3 ± 2.2* | 26.5 ± 0.9* | 30.8 ± 2.2* | 36.1 ± 2.0* | 42.1 ± 2.9* | 52.5 ± 3.3* |

*p < 0.05 peptide + transferrin versus peptide alone.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### SEQUENCE LISTING XML

### Incorporation by Reference of Sequence Listing XML:

Name of XML file: Mascarenhas Seq File 20242.xml
Date of Creation: Jan. 9, 2024
Size of XML file in bytes: 3 KB

What is claimed is:

1. A method for improving the therapeutic index of a small-molecule nuclear receptor ligand in the treatment of the peripheral immune tissues of a mammalian subject comprising:
(a) chemical synthesis of a peptide 20-60 amino acids long comprising an amino acid sequence corresponding to SEQ ID NO:1 or SEQ ID NO:2;
(b) covalent attachment of the small-molecule nuclear receptor ligand to the synthetic peptide;
(c) purification of the ligand-modified peptide;
(d) mixing of the purified ligand-modified peptide with purified recombinant human transferrin at a peptide-to-transferrin molar ratio of between 0.01 and 10;
(e) trapping of the peptide-transferrin mixture in a biodegradable polymer formulation;
(f) suspension of the formulation in a suitable pharmaceutical diluent containing at least 0.001% high-molecular-weight hyaluronic acid;
(g) administration of the formulation to a mammalian subject via a transdermal or intradermal route;
thereby achieving at least a two-fold expansion of therapeutic index of the small-molecule nuclear receptor ligand-modified peptide in the treatment of the peripheral immune system compared to a molar equivalent dose of the same small-molecule nuclear receptor ligand when administered alone by the same route.

2. The method according to claim 1 wherein the small-molecule nuclear receptor ligand is selected from a group consisting of HX600, HX630, celastrol, LG100754, GW7647, tamibarotene, GW3965, AM580, palovarotene, adapalene, bexarotene, capric acid, fenofibric acid, GW4064, sobetirol and GW501516.

3. The method according to claim 1 wherein the biodegradable polymer is poly-lactic-co-glycolic acid or polycaprolactone.

4. The method according to claim 1 wherein the diluent contains at least 0.001% of a botanical compound selected from a group consisting of resveratrol, quercetin, menthol, anisic acid, verbenone, eugenol, linalool, ferulic acid, glycyrrhizic acid, levulinic acid, alkali salt of olive oil and alkali salt of coconut oil.


SEQUENCE LISTING (2 sequences)

SEQ ID NO: 1 - protein, 21 aa, synthetic construct
KGFYKKKQCRPSKGRKRGFCW

SEQ ID NO: 2 - protein, 21 aa, synthetic construct
KGFYKRKQCKPSRGRKRGICW